(19) United States
(12) Patent Application Publication
(10) Pub. No.: US 2026/0016385 A1
(43) Pub. Date: Jan. 15, 2026

(54) APPLICABILITY REGULATION METHOD FOR CEMENT-BASED MATERIALS

(71) Applicant: SHANDONG UNIVERSITY, Jinan (CN)

(72) Inventors: Fengling ZHANG, Jinan (CN); Kai PANG, Jinan (CN); Jinghang ZHANG, Jinan (CN); Jinglong LI, Jinan (CN); Chenxu YAN, Jinan (CN); Xiufang ZHENG, Jinan (CN); Tongchun SU, Jinan (CN); Xiaoyu LI, Jinan (CN); Yupeng CUI, Jinan (CN); Xiangyu MENG, Jinan (CN); Min TANG, Jinan (CN)

(21) Appl. No.: 19/267,500

(22) Filed: Jul. 12, 2025

(30) Foreign Application Priority Data
Jul. 12, 2024 (CN) 2024109309245

(51) Int. Cl. G01N 3/48; C04B 14/04; C04B 14/48; G01N 3/30; G01N 33/38

(52) U.S. Cl. CPC G01N 3/48; C04B 14/048; C04B 14/48; G01N 3/30; G01N 33/383; C04B 2201/52

(57) ABSTRACT

The provided is an applicability regulation method for cement-based materials including: obtaining respectively Rockwell hardnesses and equivalent lengths of each phase in cement-based materials; determining effective hardnesses of the cement-based materials through weighted calculation; measuring Rockwell hardnesses of non-rigid projectiles as effective hardnesses; calculating the effective hardnesses of the non-rigid projectiles and relative hardnesses of the cement-based materials; establishing relationship curves between penetration resistances and the relative hardnesses, as well as between projectile deformation degrees and the relative hardnesses by measuring penetration resistance and a projectile deformation degree for various cement-based material and non-rigid projectile combinations with different relative hardness; confirming a relative hardness application value, and calculating an effective hardness application value of the cement-based material based on the effective hardness of the non-rigid projectile; and regulating a volume ratio of each phase of the cement-based material to make the effective hardness consistent with the effective hardness application value.

### CROSS-REFERENCE TO RELATED APPLICATIONS

The present disclosure claims priority to Chinese Patent Application No. 202410930924.5 filed to China National Intellectual Property Administration on Jul. 12, 2024, and entitled “Method for Evaluating Penetration Resistance of Cement-based Material Against Non-rigid Projectiles”, which is incorporated herein by reference in its entirety.

### TECHNICAL FIELD

The present disclosure belongs to the technical field of cement-based material applicability regulation, and relates to an applicability regulation method for cement-based materials.

### BACKGROUND

Information of the background is merely disclosed to increase the understanding of the overall background of the present disclosure, but is not necessarily regarded as acknowledging or suggesting, in any form, that the information constitutes the prior art known to those skilled in the art.

During the penetration of a non-rigid projectile into a target material, the target material surrounding the projectile is subjected to high-intensity pressure, causing deformation of the penetrating projectile under such high pressure. The degree of projectile deformation generally depends on the impact velocity and the relative characteristics between the projectile and the target. For a given target and projectile, three penetration regimes emerge with the increase of projectile velocity, including a non-deformable penetration regime (velocity less than 1000 m/s), a semi-hydrodynamic penetration regime (velocity between 1000 m/s and 3000 m/s), and a hydrodynamic penetration regime (velocity greater than 3000 m/s). At present, research on deformable penetration mechanisms has primarily focused on standard-strength concrete. For the deformable penetration mechanism, existing studies typically investigate the influence of velocity on penetration performance for a given projectile-target combination. However, in addition to the impact velocity, the projectile penetration ability also depends on the relative characteristics between the projectile and the target. For cement-based material targets, the influence of these relative characteristics at a given impact velocity remains not readily available in the literature.

As shown in FIG. 1, the normalized penetration depth (normalized penetration depth=penetration depth/impact velocity) generally increases with the relative strength (the ratio of the strength of the projectile to the strength of the target material) until a particular degree is reached. However, beyond the particular degree, the normalized penetration depth decreases sharply. In this case, the relative strength has lost its clear association relationship with the penetration resistance, and the penetration resistance cannot be evaluated based on the magnitude of relative strength. Therefore, there is an urgent need to propose a method for evaluating the penetration resistance of the cement-based material that comprehensively considers the compositional characteristics of both the projectile and the target material.

### SUMMARY

Aiming at the limitations of the existing technology, the objective of the present disclosure is to provide an applicability regulation method for cement-based materials based on relative hardness. The damage degree of the projectile is investigated based on the changes in the length, diameter, and mass of the projectile, and the corresponding deformation mechanism is briefly discussed to determine the appropriate relative characteristics. The Rockwell hardness can be used to quickly estimate the penetration depth of the anti-deformation projectile impact. According to the estimated penetration depth parameter value, the volume ratio of each phase material in the cement-based material with unknown non-rigid projectile penetration performance is confirmed and regulated. A cement-based material for the intended use is prepared based on the volume ratio of each phase material contained in the cement-based material that meets the requirement on the non-rigid projectile penetration resistance.

In order to realize the above objective, the present disclosure adopts the following technical solution:

An applicability regulation method for cement-based materials is provided, including:
S1: considering a cement-based material as a multiphase material, respectively measuring a Rockwell hardness of each phase, and calculating an equivalent length of each phase based on a volume ratio of each phase;
S2: determining an effective hardness of the cement-based material through weighted calculation based on the Rockwell hardness of each phase and the equivalent length of each phase;
S3: preparing a non-rigid projectile using a single-phase material as a parent material, and measuring a Rockwell hardness of the parent material as an effective hardness of the non-rigid projectile;
S4: calculating a ratio of the effective hardness of the non-rigid projectile to the effective hardness of the cement-based material to obtain relative hardness;
S5: measuring penetration resistance and a projectile deformation degree for various cement-based material and non-rigid projectile combinations with different relative hardness, and respectively establishing relationship curves between the penetration resistance and the relative hardness as well as between the projectile deformation degree and the relative hardness; using a relative hardness value corresponding to an intersection point of the relationship curve between the penetration resistance and the relative hardness, as well as the relationship curve between the projectile deformation degree and the relative hardness as a relative hardness application value in an applicability regulation for the cement-based materials;
S6: considering a to-be-measured cement-based material with unknown non-rigid projectile penetration resistance as a multiphase material, respectively measuring a Rockwell hardness of each phase, and calculating an equivalent length of each phase based on a volume ratio of each phase; determining an effective hardness of the to-be-measured cement-based material through weighted calculation based on the Rockwell hardness of each phase and the equivalent length of each phase;
S7: calculating the relative hardness application value of the cement-based material using the relative hardness application value based on the known effective hardness of the non-rigid projectile; regulating the volume ratio of each phase in the to-be-measured cement-based material; repeating S2 to S4 and comparing the relative hardness of the to-be-measured cement-based material with the calculated relative hardness application value; and
S8: when the relative hardness of the to-be-measured cement-based material is consistent with the relative hardness application value, preparing the cement-based material based on the volume ratio of each phase material in the to-be-measured cement-based material, and using the cement-based material in the construction of a predefined structure.

Optionally, in S1, considering the cement-based material as a three-phase material composed of a mortar matrix phase, a coarse aggregate phase and a fiber phase, obtaining a sample of the cement-based material, and measuring a Rockwell hardness of a surface of the sample as a Rockwell hardness of the mortar matrix phase; obtaining a coarse aggregate sample of the cement-based material, and measuring a Rockwell hardness of a surface of the coarse aggregate sample; and obtaining a fiber sample of the same material as fibers in the cement-based material, and measuring a Rockwell hardness of the fiber sample.

Optionally, the mortar matrix phase includes one or more of cement, silica fume, and fly ash; the coarse aggregate phase includes 5-16 mm rock aggregate; and the fiber phase is one or more of straight copper-coated steel fibers, hooked-end copper-coated steel fibers, and organic fibers.

Optionally, in S1, a volume ratio of the mortar matrix phase, the coarse aggregate phase and the fiber phase is calculated, and an equivalent length of the mortar matrix phase, an equivalent length of the coarse aggregate phase, and an equivalent length of the fiber phase are respectively obtained.

Optionally, in S1, the equivalent length of each phase is obtained through a cube root of the volume ratio of each phase, and the equivalent length of the fiber phase is corrected according to the type of fibers.

Optionally, in S2, in a weighted calculation method, a weight of an effective hardness of each phase is equal to a ratio of the equivalent length of the phase to a sum of the equivalent lengths of all phases.

Optionally, in S3, a sample of the non-rigid projectile parent material is obtained and the Rockwell hardness of the surface of the sample is measured.

Optionally, in S5, an impact velocity is 390-430 m/s.

Optionally, in S5, the penetration resistance includes normalized penetration depth and crater diameter;
a relationship between the normalized penetration depth Y_{penetration depth} and the relative hardness REH is:

$${Y}_{penetration depth} = 138.727 - \frac{1045.12}{1 + {( \frac{REH}{0.044} )}^{0.646}} ;$$

a relationship between the crater diameter Y_{crater diameter} and the relative hardness REH is:

$${Y}_{crater diameter} = 74.56 - \frac{19893.505}{1 + {( \frac{REH}{0.120} )}^{0.120}} ;$$

Optionally, in S5, the projectile deformation degree includes normalized projectile length change, normalized projectile diameter change, and normalized projectile mass change;
a relationship between the normalized projectile mass change γₘ and the relative hardness REH is:

$${γ}_{m} = 1.229 + \frac{22859.691}{1 + {( \frac{REH}{0.361} )}^{9.307}} ;$$

a relationship between the normalized projectile length change γₗ and the relative hardness REH is:

$${γ}_{l} = 20.197 - \frac{217.6}{1 + {( \frac{REH}{0.947} )}^{8.544}} ;$$

a relationship between the normalized projectile diameter change γ_{d} and the relative hardness REH is:

$${γ}_{d} = 13.018 + \frac{6218.941}{1 + {( \frac{REH}{0.612} )}^{6.547}} .$$

The present disclosure has the following beneficial effects:

1. The present disclosure provides a method for evaluating penetration resistance of a cement-based material against non-rigid projectiles based on relative hardness. The established characterization system can accurately predict the penetration resistance of cement-based materials with different material components and complex properties under the action of non-rigid projectiles, including penetration depth and crater diameter, as well as the damage situation of the projectile, such as changes in projectile length, projectile diameter, and projectile mass. Compared to the traditional penetration experiment, the method provided in the present disclosure can not only quickly evaluate the penetration resistance of the cement-based material, but also significantly improve the reliability and repeatability of testing due to its minimal damage to the sample and ability to maintain the integrity of the sample.

2. The present disclosure delves into the relative hardness and strength difference between the projectile and the target, as well as the influence on the damage degree of the two in the interaction process. Through the detailed analysis of the penetration of the non-rigid projectile into the cement-based material, the penetration mode can be divided into two stages. When the relative hardness value is small, the main manifestation of penetration damage is the deformation of the projectile, and the damage degree of the projectile becomes more severe as the relative hardness decreases. When the relative hardness value is large, the main manifestation of penetration damage is the damage of the target, and the damage of the target becomes more obvious as the relative hardness increases. By analyzing the two stages, the dynamic interaction and damage mechanism between the non-rigid projectile and the cement-based material target can be more deeply understood. This in-depth analysis provides a scientific basis for the design and material selection of the protective structure, which can promote the development of related engineering applications.

### BRIEF DESCRIPTION OF THE DRAWINGS

The drawings of the description, which form a part of the present disclosure, are intended to provide a further understanding of the present disclosure. The exemplary examples of the present disclosure and their descriptions are intended to describe the present disclosure, instead of constituting any improper limitation on the present disclosure.

FIG. 1 is a schematic diagram showing a relationship between normalized penetration depth and relative strength of a cement-based material in the background of the present disclosure.

FIG. 2 is a schematic diagram showing a relationship between relative hardness and normalized penetration depth in Example 1 of the present disclosure.

FIG. 3 is a schematic diagram showing a relationship between relative hardness and crater diameter in Example 1 of the present disclosure.

FIG. 4 is a schematic diagram showing a relationship between relative hardness and normalized penetration depth in Example 1 of the present disclosure.

FIG. 5 is a schematic diagram showing a relationship between relative hardness and projectile diameter change in Example 1 of the present disclosure.

FIG. 6 is a schematic diagram showing a relationship between relative hardness and projectile mass change in Example 1 of the present disclosure.

FIG. 7 is a schematic diagram showing a relationship between deformation ability and penetration ability of a cement-based material in Example 1 of the present disclosure.

FIG. 8 is a schematic diagram of the size of a non-rigid projectile before penetration in a specific implementation of the present disclosure.

FIG. 9 is a schematic diagram of the remaining length of a non-rigid projectile after penetration in a specific implementation of the present disclosure.

FIG. 10 is a schematic diagram of the diameter of a non-rigid projectile after penetration in a specific implementation of the present disclosure.

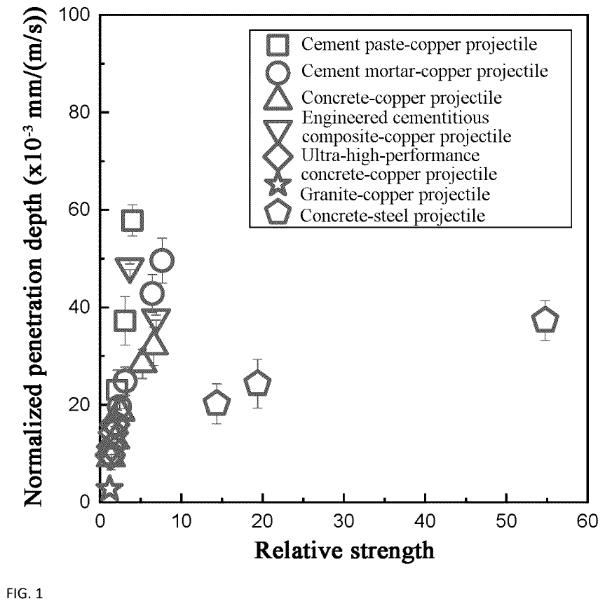

### DETAILED DESCRIPTION

It should be noted that, the following detailed descriptions are all exemplary, and are intended to provide further descriptions of the present disclosure. Unless otherwise specified, all technical and scientific terms used herein have the same meanings as those usually understood by a person of ordinary skill in the art to which the present disclosure belongs.

It should be noted that the terms used herein are merely used for describing specific implementations, and are not intended to limit exemplary implementations of the present disclosure. As used herein, the singular form is intended to include the plural form, unless the context clearly indicates otherwise. In addition, it should further be understood that terms “comprise” and/or “include” used in this specification indicate that there are features, steps, operations, devices, components, and/or combinations thereof.

An applicability regulation method for cement-based materials, including:
S1: considering a cement-based material as a multiphase material, respectively measuring a Rockwell hardness of each phase, and calculating an equivalent length of each phase based on a volume ratio of each phase;
S2: determining an effective hardness of the cement-based material through weighted calculation based on the Rockwell hardness of each phase and the equivalent length of each phase;
S3: preparing a non-rigid projectile using a single-phase material as a parent material, and measuring a Rockwell hardness of the parent material as an effective hardness of the non-rigid projectile;
S4: calculating the relative hardness of a combination of the cement-based material and the non-rigid projectile based on the effective hardness of the cement-based material and the non-rigid projectile;
S5: measuring penetration resistance and a projectile deformation degree for various cement-based material and non-rigid projectile combinations with different relative hardness by adopting fixed projectile velocity, and respectively establishing relationship curves between the penetration resistance and the relative hardness, as well as between the projectile deformation degree and the relative hardness; using a relative hardness value corresponding to an intersection point of the relationship curve between the penetration resistance and the relative hardness, as well as the relationship curve between the projectile deformation degree and the relative hardness as a relative hardness application value in an applicability regulation for the cement-based materials;
S6: considering a to-be-measured cement-based material with unknown non-rigid projectile penetration resistance as a multiphase material, respectively measuring a Rockwell hardness of each phase, and calculating an equivalent length of each phase based on a volume ratio of each phase; determining an effective hardness of the to-be-measured cement-based material through weighted calculation based on the Rockwell hardness of each phase and the equivalent length of each phase;
S7: calculating the relative hardness application value of the cement-based material using the relative hardness application value based on the known effective hardness of the non-rigid projectile; regulating the volume ratio of each phase in the to-be-measured cement-based material; repeating S2 to S4 and comparing the relative hardness of the to-be-measured cement-based material with the calculated relative hardness application value; and
S8: when the relative hardness of the to-be-measured cement-based material is consistent with the relative hardness application value, preparing the cement-based material based on the volume ratio of each phase material in the to-be-measured cement-based material, and using the cement-based material in the construction of a predefined structure.

Optionally, in S1, the cement-based material is considered as a three-phase material composed of a mortar matrix phase, a coarse aggregate phase and a fiber phase, a sample of the cement-based material is obtained, and a Rockwell hardness of a surface of the sample is measured as a Rockwell hardness Hₘₐₜᵣᵢₓ of the mortar matrix phase. A coarse aggregate sample of the cement-based material is obtained, and the Rockwell hardness H_{coarse aggregate} of the surface of the coarse aggregate sample is measured. A fiber sample of the same material as fibers in the cement-based material is obtained, and the Rockwell hardness H_{fiber} of the fiber sample is measured.

Optionally, the mortar matrix phase includes one or more of cement, silica fume, and fly ash. The coarse aggregate phase includes 5-16 mm rock aggregate. The fiber phase is one or more of straight copper-coated steel fibers, hooked-end copper-coated steel fibers, and organic fibers.

Optionally, in S1, a volume ratio of the mortar matrix phase, the coarse aggregate phase and the fiber phase is calculated, and an equivalent length of the mortar matrix phase, an equivalent length of the coarse aggregate phase, and an equivalent length of the fiber phase are respectively obtained. An equivalent length of the mortar matrix phase is denoted as lₘₐₜᵣᵢₓ. The equivalent length of coarse aggregate phase is denoted as l_{coarse aggregate}. The equivalent length of the fiber phase is denoted as l_{fiber}.

Optionally, in S1, the equivalent length of each phase is obtained through a cube root of the volume ratio of each phase, and the equivalent length of the fiber phase is corrected according to the type of fibers.

Optionally, in S1, the equivalent length of each phase is calculated according to the following formulas:

$${l}_{coarse aggregate} = \sqrt[3]{{V}_{coarse aggregate}} ;$$

$${l}_{matrix} = \sqrt[3]{{V}_{matrix}} ;$$

$${l}_{fiber} = \sqrt[3]{k {V}_{fiber}} ;$$

$${V}_{coarse aggregate} + {V}_{matrix} + {V}_{fiber} = 100 \% ;$$

where V_{coarse aggregate} is a volume ratio of the coarse aggregate phase in the cement-based material;
Vₘₐₜᵣᵢₓ is a volume ratio of the mortar matrix phase in the cement-based material;
V_{fiber} is a volume ratio of the fiber phase in the cement-based material;
the value of k is selected according to the type of fibers; when the fibers are straight copper-coated steel fibers, the value of k is taken as 1.0; when the fibers are hooked-end copper-coated steel fibers, the value of k is taken as 1.2; and when the fibers are organic fibers, the value of k is taken as 0.9.

Optionally, in S2, in a weighted calculation method, a weight of an effective hardness of each phase is equal to a ratio of the equivalent length of the phase to a sum of the equivalent lengths of all phases.

Optionally, the effective hardness H_{effective} of the cement-based material target is calculated according to the following formula:

$${H}_{effective} = \frac{{l}_{coarse aggregate}}{{l}_{coarse aggregate} + {l}_{matrix} + {l}_{fiber}} {H}_{coarse aggregate} + \frac{{l}_{matrix}}{{l}_{coarse aggregate} + {l}_{matrix} + {l}_{fiber}} {H}_{matrix} + \frac{{l}_{fiber}}{{l}_{coarse aggregate} + {l}_{matrix} + {l}_{fiber}} {H}_{fiber} .$$

Optionally, in S3, a sample of the non-rigid projectile parent material is obtained, the Rockwell hardness H_{projectile} of the surface of the sample is measured, and the method for measuring the Rockwell hardness is the same as the method for measuring the Rockwell hardness in S1.

Optionally, in S4, the relative hardness REH is the ratio of the effective hardness of the non-rigid projectile to the effective hardness of the cement-based material.

Optionally, the relative hardness REH is calculated according to the following formula:

$$REH = \frac{{H}_{projectile}}{{H}_{effective}} .$$

Optionally, in S5, the penetration resistance includes normalized penetration depth.

Optionally, in S5, the projectile deformation degree includes normalized projectile length change γₗ, normalized projectile diameter change γ_{d}, and normalized projectile mass change γₘ,
where

$${γ}_{l} = \vert \frac{{l}_{0} - {l}_{r}}{{l}_{0}} \vert / v ;$$

$${γ}_{d} = \vert \frac{{d}_{0} - {d}_{r}}{{d}_{0}} \vert / v ;$$

$${γ}_{m} = \vert \frac{{m}_{0} - {m}_{r}}{{m}_{0}} \vert / v .$$

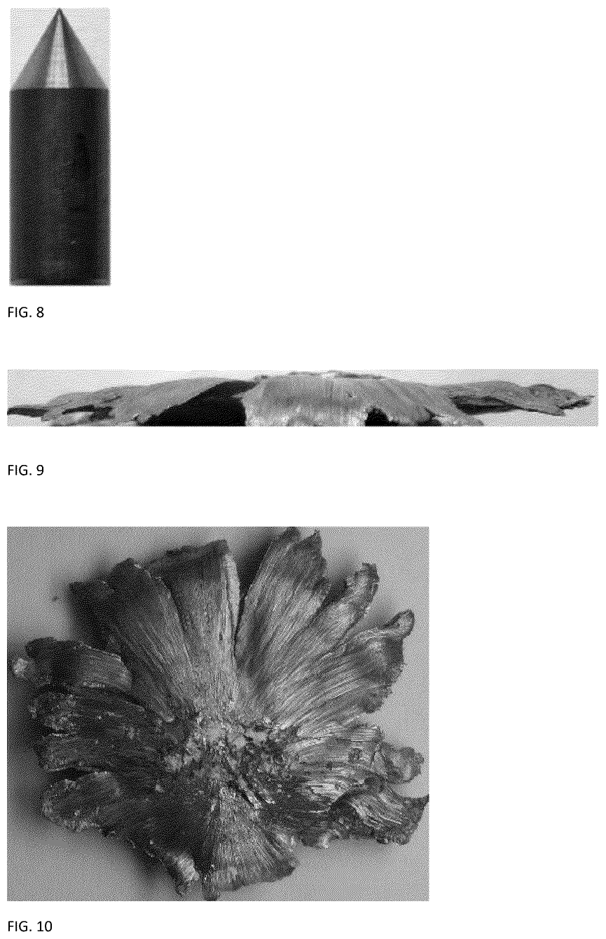

As shown in FIG. 8, l₀ and d₀ are respectively the initial length and diameter of the projectile. As shown in FIG. 9, lᵣ represents the remaining length of the projectile after an experiment. As shown in FIG. 10, dᵣ is the diameter of the projectile after the experiment and is the average of the two lengths (dᵣ₁ and dᵣ₂) in an orthogonal direction, that is,

$${d}_{r} = \frac{{d}_{r 1} + {d}_{r 2}}{2} ;$$

m₀ is the initial mass of the projectile; mᵣ is the remaining mass of the projectile after the experiment; and v is the impact velocity of the projectile.

### Example 1

1. Cement paste, cement mortar, concrete materials, engineered cementitious composites, and ultra-high-performance concrete with different component ratios were adopted to test their compressive strength. Cement-based material targets with a size of 300*170*150 mm³ (where 150 mm is the thickness along the ballistic direction) were prepared.

The cement paste was numbered as CP-1 to CP-3. Since a coarse aggregate phase and a fiber phase were not contained, the effective lengths of the coarse aggregate phase and the fiber phase were both 0.

The cement mortar was numbered as CM-1 to CM-6. Since a coarse aggregate phase and a fiber phase were not contained, the effective lengths of the coarse aggregate phase and the fiber phase were both 0.

The concrete materials were numbered as CC-1 to CC-5. Components included cement mortar and coarse aggregate, including or excluding fibers.

The engineered cementitious composites were numbered as ECC-1 to ECC-2. Components included water, silica fume, fly ash, fibers, a water reducing agent, and river sand, where river sand was fine aggregate and belonged to a component of cement mortar. Coarse aggregate was not included, so the effective length of the coarse aggregate phase was 0.

The ultra-high-performance concrete was numbered as UHPC-1 to UHPC-4. Components included cement mortar matrix, coarse aggregate, and fibers.

In addition, granite targets with the same material as the coarse aggregate were prepared to test their compressive strength. Granite did not include cement mortar and fibers, so the effective lengths of the matrix phase and the fiber phase were 0.

Projectiles with the same shape were respectively prepared using ASSAB XW-42 high-strength alloy steel or copper.

The yield strength of ASSAB XW-42 high-strength alloy steel was 2150-2200 MPa, the peak strength was 2950-3100 MPa, and the effective hardness was 97.2 HR15T.

The yield strength of copper was 275 MPa, the peak strength was 290 MPa, and the effective hardness was 78.4 HR15T.

2. The Rockwell hardness of each phase in the cement-based materials and the Rockwell hardness of the projectiles were measured, including:

The cement-based materials were cast into cubic blocks of 100*100*100 mm³, curing was performed, then polishing and cleaning were performed, then straight lines perpendicular to the edges were equidistantly drawn on formwork surfaces, and each formwork surface was divided into 25 square test areas of 20*20 mm². Each test block had four formwork surfaces available for measurement, and at least nine measurement points were selected for measurement on each formwork surface. To avoid the influence of the edge effect, the peripheral 16 square test areas in contact with the edge were discarded, and the middle points of the middle nine square areas were selected as the pressure measurement point areas.

The Rockwell hardness of the test blocks was measured using a Rockwell hardness tester, a steel ball with a diameter of 1.5875 mm was selected as an indenter, the initial test force was 29.42 N, and the total test force was 147.1 N. The baseline indentation depth h₀ was recorded after the initial test force was reached and maintained for 15 s, and then the test force was increased to the total test force and maintained for 15 s to measure the final indentation depth h₁. By calculating the residual indentation depth h (calculation formula: h=h₁−h₀), the Rockwell hardness H of the mortar matrix in concrete could be further calculated.

$$H = 100 - \frac{h}{0.001} .$$

Similarly, granite samples were measured according to the above method.

In the testing method for the Rockwell hardness of the fiber phases, considering that the fiber diameter was relatively small (usually 0.2 mm), the Rockwell hardness of all fiber phases was measured from fiber parent material test blocks. The fiber parent material test blocks were test blocks obtained by cutting fiber raw materials, which had the same performance as the fiber phases but were sized for ease of measurement.

In the measurement method for the Rockwell hardness of the projectiles, considering that the volume of the projectiles is small, the Rockwell hardness was measured from projectile parent material test blocks. The projectile parent material test blocks were test blocks obtained by cutting projectile raw materials. Since the projectiles were of a single-phase material, the Rockwell hardness could be equivalent to the effective hardness of the projectiles.

3. The equivalent length of each phase in the cement-based materials was obtained and the effective hardness was calculated, including:

Each component phase in the cement-based materials was equivalent to a cube, the volume of the cube was determined based on the volume ratio of each phase. The cube root of the volume ratio (percentage) of the cube was calculated to obtain the side length of the cube, which served as the equivalent length of the phase.

The calculation method for the equivalent length of the fiber phase was as follows: the equivalent cube volume ratio of the fiber phase was multiplied by a correction factor k, and then the cube root of the volume corrected by the correction factor k was calculated to obtain the equivalent length of the fiber phase. This was because the different materials, shapes, and structures of fibers in fiber-reinforced cement-based materials could have varying degrees of influence on the hardness and penetration resistance of the fiber-reinforced cement-based materials, and it was difficult to measure these influences solely through the volume ratio. Specifically, when straight copper-coated steel fibers were used, k was taken as 1.0; when hooked-end copper-coated steel fibers were used, k was taken as 1.2; and when organic fibers were used, k was taken as 0.9.

The effective hardness of the cement-based materials was calculated. The effective hardness of the cement-based materials was denoted as H_{effective}. The weighting calculation method was as follows:

$${H}_{effective} = \frac{{l}_{coarse aggregate}}{{l}_{coarse aggregate} + {l}_{matrix} + {l}_{fiber}} {H}_{coarse aggregate} + \frac{{l}_{matrix}}{{l}_{coarse aggregate} + {l}_{matrix} + {l}_{fiber}} {H}_{matrix} + \frac{{l}_{fiber}}{{l}_{coarse aggregate} + {l}_{matrix} + {l}_{fiber}} {H}_{fiber} ;$$

where

$${l}_{coarse aggregate} = \sqrt[3]{{V}_{coarse aggregate}}$$

was the equivalent length of the coarse aggregate in the cement-based material;

$${l}_{matrix} = \sqrt[3]{{V}_{matrix}}$$

was the equivalent length of the mortar matrix in the cement-based material;

$${l}_{fiber} = \sqrt[3]{k {V}_{fi ber}}$$

was the equivalent length of the fibers in the cement-based material, the value of k was selected according to the type of fibers; when the fibers were straight copper-coated steel fibers, the value of k was taken as 1.0; when the fibers were hooked-end copper-coated steel fibers, the value of k was taken as 1.2; when the fibers were organic fibers, the value of k was taken as 0.9;
H_{coarse aggregate} was the Rockwell hardness value of the coarse aggregate in the cement-based material;
Hₘₐₜᵣᵢₓ was the Rockwell hardness value of the mortar matrix in the cement-based material; and
H_{fiber} was the Rockwell hardness value of the fibers in the cement-based material.

3. The relative hardness of combinations of the cement-based materials and the non-rigid projectiles was calculated based on the effective hardness of the cement-based materials of different materials and the effective hardness of the non-rigid projectiles of different materials. An example was as follows:

For the effective hardness of the target material, taking concrete C-5 as an example, the Rockwell hardness and volume ratio of each phase were as shown in Table 1, where the Rockwell hardness was measured under HR15T conditions.

TABLE 1
Rockwell hardness and volume ratio of each phase

| | Rockwell hardness | | | Volume content | | |
|---|---|---|---|---|---|---|
| | Mortar | Coarse | Steel | Mortar | Coarse | Steel |
| | matrix | aggregate | fibers | matrix | aggregate | fibers |
| Concrete C-5 | 69.1 | 93.7 | 80.3 | 64.078% | 35.422% | 0.500% |
| (containing | | | | | | |
| coarse aggregate) | | | | | | |

By substituting the data in Table 1 into the weighted calculation method in Example 1, the calculation result of the effective hardness H_{effective} was the effective hardness H_{target} of the target:

$$\frac{\sqrt[3]{0.35422}}{\sqrt[3]{0.35422} + \sqrt[3]{0.64078} + \sqrt[3]{0.005}} * 93.7 + \frac{\sqrt[3]{0.64078}}{\sqrt[3]{0.35422} + \sqrt[3]{0.64078} + \sqrt[3]{0.005}} * 6 9.1 + \frac{\sqrt[3]{0.005}}{\sqrt[3]{0.35422} + \sqrt[3]{0.64078} + \sqrt[3]{0.005}} = 80.2 .$$

When ASSAB XW-42 high-strength alloy steel was used for the projectile, the relative hardness was:

$$REH = \frac{{H}_{projectile}}{{H}_{effective}} = \frac{97.2}{802} = 1.2 \; 12 ;$$

when copper was used for the projectile, the relative hardness was:

$$REH = \frac{{H}_{projectile}}{{H}_{effective}} = \frac{78.4}{80.2} = 0.9776 .$$

4. Penetration resistance testing was performed. A high-speed projectile penetration apparatus was set up. A ballistic smoothbore gun was used to launch the ASSAB XW-42 high-strength alloy steel projectile or the copper projectile. The projectile was a conical bullet with a diameter of 8 mm and impact velocity of 420 m/s. The launch direction was parallel to the ballistic thickness direction of the target. The penetration crater was located at the center of a target plane. In the process, a laser velocimetry system was used to measure the velocity of the projectile before and after launch, and a high-speed camera was used to monitor whether the ballistic direction was perpendicular to the target, so as to eliminate atypical test results caused by launch angle deviation.

After the penetration testing was completed, the damage degree of the cement-based material target under the action of the high-speed projectile was measured through the penetration depth (i.e., the distance from the position that the projectile penetrated the target to the deepest point) and the crater diameter (the diameter of an equivalent circle with the same area as the crater), and the normalized penetration depth Y_{penetration depth} was calculated through the penetration depth. The impact velocity was obtained by measuring the projectile velocity after launch using the laser velocimetry system.

As shown in Table 2, it shows the deformation of the targets under the penetration of the ASSAB XW-42 high-strength alloy steel projectiles.

TABLE 2
deformation of targets under penetration of ASSAB
XW-42 high-strength alloy steel projectiles

| | | | | Normalized | |
|---|---|---|---|---|---|
| | Compressive | Effective | | penetration | Crater |
| | strength | hardness | Relative | depth (*10⁻³ | diameter |
| S/N | (MPa) | (HR15T) | hardness | mm/(m/s)) | (mm) |
| CC-2 | 55.2 | 72.2 | 1.3463 | 37.3 | 58.5 |
| CC-4 | 156.0 | 85.6 | 1.1355 | 24.3 | 52.1 |
| CC-5 | 210.2 | 87.0 | 1.1172 | 20.2 | 49.2 |

As shown in Table 3, it shows the deformation of the targets under the penetration of the copper projectiles.

TABLE 3
deformation of targets under penetration of copper projectiles

| | | | | Normalized | Crater |
|---|---|---|---|---|---|
| | Compressive | Effective | | penetration | dia- |
| | strength | hardness | Relative | depth (*10⁻³ | meter |
| S/N | (MPa) | (HR15T) | hardness | mm/(m/s)) | (mm) |
| CP-1 | 72.4 | 39.5 | 1.9848 | 57.8 | 75.6 |
| CP-2 | 93.8 | 52.8 | 1.4848 | 37.2 | 66.9 |
| CP-3 | 136.0 | 73.6 | 1.0652 | 23.0 | 49.5 |
| CM-1 | 37.9 | 46.2 | 1.6970 | 49.6 | 66.9 |
| CM-2 | 45.0 | 50.1 | 1.5649 | 42.8 | 56.2 |
| CM-3 | 93.4 | 69.3 | 1.1313 | 24.8 | 48.8 |
| CM-4 | 120 | 74.2 | 1.0566 | 19.6 | 44.9 |
| CM-5 | 146.9 | 76.2 | 1.0289 | 16.0 | 40.4 |
| CM-6 | 190.9 | 80.0 | 0.9800 | 9.6 | 26.4 |
| CC-1 | 43.8 | 68.2 | 1.1496 | 32.0 | 50.4 |
| CC-2 | 55.2 | 72.2 | 1.0859 | 28.4 | 50.7 |
| CC-3 | 118.1 | 80.2 | 0.9776 | 18.6 | 41.7 |
| CC-4 | 156.0 | 85.6 | 0.9159 | 12.7 | 36.5 |
| CC-5 | 210.2 | 87.0 | 0.9011 | 9.0 | 26.7 |
| ECC-1 | 42 | 54.1 | 1.4492 | 37.9 | 43.3 |
| ECC-2 | 78.8 | 47.4 | 1.6540 | 48.3 | 44.6 |
| UHPC-1 | 161.0 | 76.8 | 1.0208 | 15.9 | 38.2 |
| UHPC-2 | 175.6 | 77.9 | 1.0064 | 14.3 | 38.2 |
| UHPC-3 | 202.3 | 83.4 | 0.9400 | 11.4 | 31.4 |
| UHPC-4 | 229.4 | 80.5 | 0.9739 | 9.7 | 33.3 |
| Granite | 238.5 | 93.7 | 0.8367 | 2.7 | 11.3 |

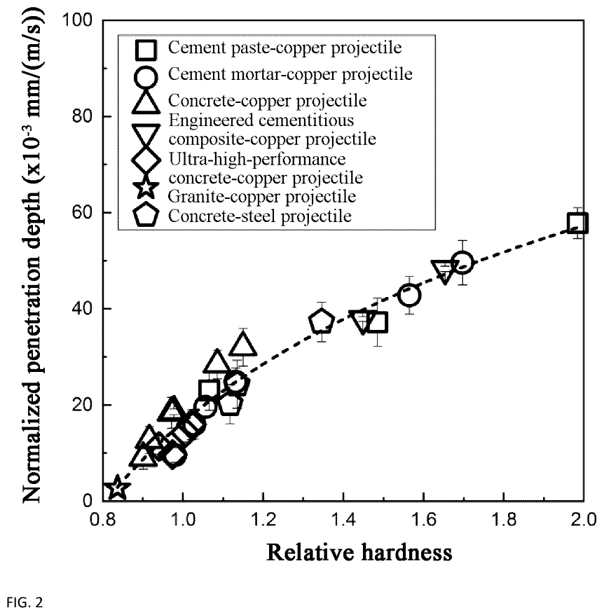

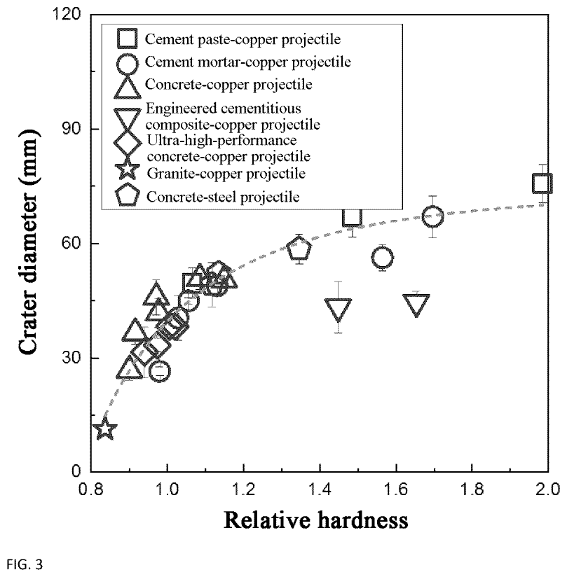

According to the data in Table 2 and Table 3, statistical processing was performed to obtain a relationship between the relative hardness and the normalized penetration depth as shown in FIG. 2, and a relationship between the relative hardness and the crater diameter as shown in FIG. 3. It can be seen that there is a clear correlation between the relative hardness and the penetration resistance, which can be evaluated based on the relative hardness.

After sorting, the evaluation method for the normalized penetration depth Y_{penetration depth} was as follows:

$${Y}_{penetration depth} = 138.727 - \frac{1045.12}{1 + {( \frac{REH}{0.044} )}^{0.646}} , {R}^{2} = 0.95 .$$

After sorting, the evaluation method for the normalized crater diameter Y_{crater diameter} was as follows:

$${Y}_{crater diameter} = 74.56 - \frac{19893.505}{1 + {( \frac{REH}{0.120} )}^{0.120}} , {R}^{2} = 0.89 .$$

The deformation of the projectiles under the penetration of the ASSAB XW-42 high-strength alloy steel projectiles was as shown in Table 4, and the deformation of the projectiles under the penetration of the copper projectiles was as shown in Table 5 and Table 6.

TABLE 4
deformation of projectiles under penetration of
ASSAB XW-42 high-strength alloy steel projectiles

| | Normalized | Normalized | Normalized |
|---|---|---|---|
| | projectile | projectile | projectile |
| | mass loss | length change | diameter change |
| S/N | (*10⁻⁵(m/s)) | (*10⁻⁵(m/s)) | (*10⁻⁵(m/s)) |
| CC-2 | 2.7 | 16.3 | 0 |
| CC-4 | 5.6 | 17.5 | 0 |
| CC-5 | 9.4 | 25.1 | 0 |

TABLE 5
deformation of projectiles under penetration
of copper projectiles (I)

| | Normalized | Normalized | Normalized |
|---|---|---|---|
| | projectile | projectile | projectile |
| | mass loss | length change | diameter change |
| S/N | (*10⁻⁵(m/s)) | (*10⁻⁵(m/s)) | (*10⁻⁵(m/s)) |
| CP-1 | 0.2 | 14.7 | 17.6 |
| CP-2 | 0.2 | 45.7 | 59.5 |
| CP-3 | 1.5 | 90.4 | 230.4 |
| CM-1 | 1.3 | 12.5 | 17.1 |
| CM-2 | 1.5 | 38.6 | 66.7 |
| CM-3 | 1.6 | 56.3 | 90.3 |
| CM-4 | 1.9 | 96.4 | 201.4 |
| CM-5 | 1.8 | 110.9 | 231.2 |

TABLE 6
deformation of projectiles under penetration
of copper projectiles (II)

| | Normalized | Normalized | Normalized |
|---|---|---|---|
| | projectile | projectile | projectile |
| | mass loss | length change | diameter change |
| S/N | (*10⁻⁵(m/s)) | (*10⁻⁵(m/s)) | (*10⁻⁵(m/s)) |
| CM-6 | 5.9 | 146.9 | 388.5 |
| CC-1 | 2.1 | 78.6 | 175.4 |
| CC-2 | 2.1 | 82.9 | 161.8 |
| CC-3 | 2.3 | 92.6 | 246.8 |
| CC-4 | 3.7 | 109.0 | 301.9 |
| CC-5 | 7.6 | 147.3 | 400.8 |
| ECC-1 | 1.0 | 24.7 | 27.0 |
| ECC-2 | 0.4 | 23.4 | 42.3 |
| UHPC-1 | 1.6 | 95.0 | 249.5 |
| UHPC-2 | 2.7 | 113.5 | 302.3 |
| UHPC-3 | 2.7 | 133.9 | 443.7 |
| UHPC-4 | 5.3 | 120.2 | 372.4 |
| Granite | 10.5 | 195.5 | 754.8 |

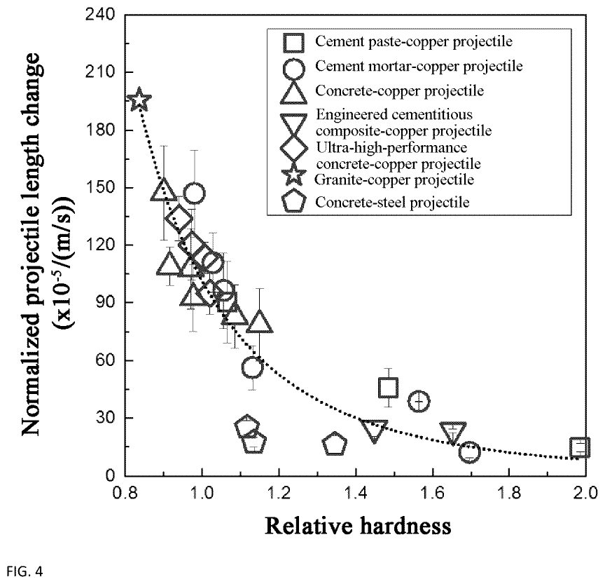

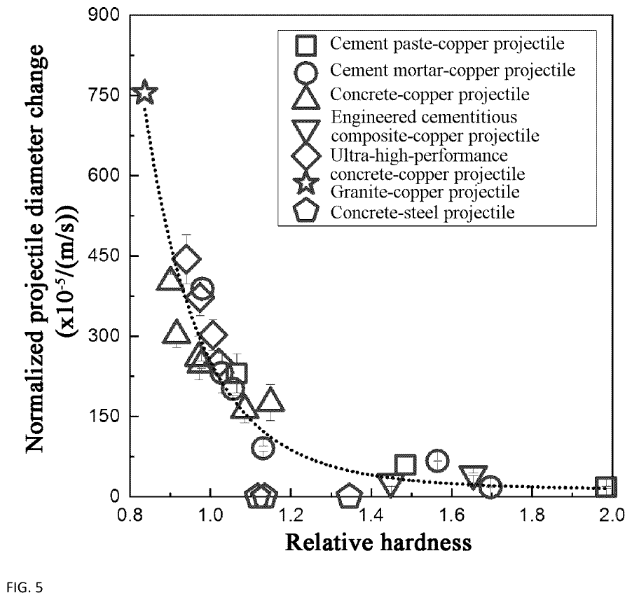

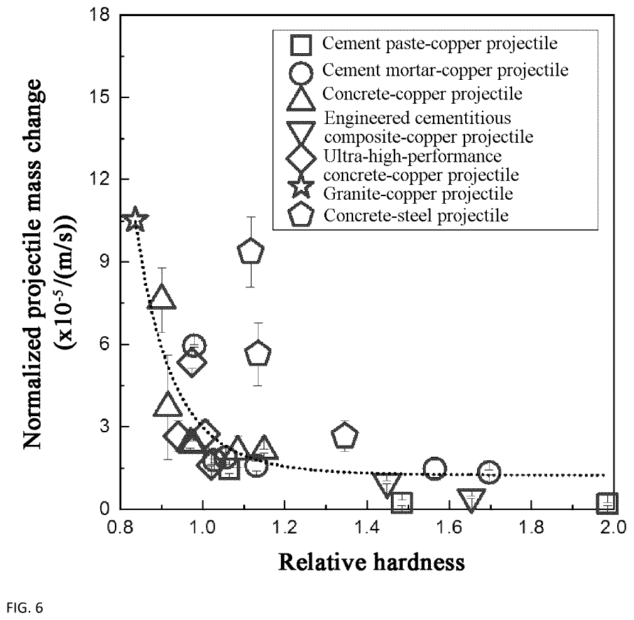

According to the data in Table 4, Table 5, and Table 6, statistical processing was performed to obtain a relationship between the relative hardness and the projectile mass loss as shown in FIG. 4, a relationship between the relative hardness and the projectile length loss as shown in FIG. 5, and a relationship between the relative hardness and the projectile diameter change as shown in FIG. 6. It can be seen that there is a clear correlation between the relative hardness and the projectile deformation ability, which can be evaluated based on the relative hardness.

After sorting, the evaluation method for the normalized projectile mass change γₘ was as follows:

$${γ}_{m} = 1.229 + \frac{22859.691}{1 + {( \frac{REH}{0.361} )}^{9.307}} , {R}^{2} = 0.8 .$$

After sorting, the evaluation method for the normalized projectile length change γₗ was as follows:

$${γ}_{l} = 20.197 - \frac{217.6}{1 + {( \frac{REH}{0.947} )}^{8.544}} , {R}^{2} = 0.88 .$$

After sorting, the evaluation method for the normalized projectile diameter change γ_{d} was as follows:

$${γ}_{d} = 13.018 + \frac{6218.941}{1 + {( \frac{REH}{0.612} )}^{6.547}} , {R}^{2} = 0.88 .$$

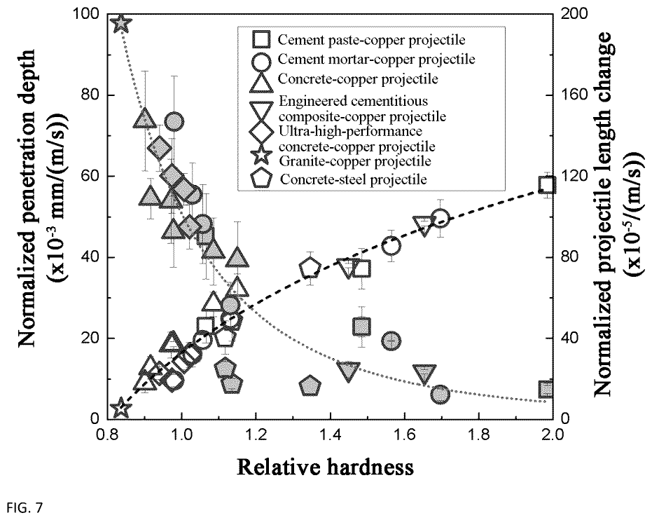

FIG. 7 shows the association relationship between the normalized penetration depth and the normalized projectile length change and the relative hardness. The point values of the normalized penetration depth are represented by hollow icons, and correspond to the vertical axis on the left. The normalized projectile length changes are represented by the shaded icons, and correspond to the vertical axis on the left. It can be seen that there is a clear association relationship between the two and the relative hardness. Moreover, through experimental testing, the penetration mode can be divided into two stages based on the level of relative hardness values. As shown in FIG. 7, the intersection point of the normalized penetration depth curve and the normalized projectile length change curve (in the figure, the horizontal axis of the intersection point is “relative hardness”=1.2) can be used. When the relative hardness value is less than 1.2, the main manifestation of penetration damage is the deformation of the projectile (projectile mass change, projectile length change, and projectile diameter change), and the projectile damage degree becomes more severe as the relative hardness decreases. When the relative hardness value is greater than 1.2, the main manifestation of penetration damage is the damage of the target (penetration depth and crater diameter), and the damage of the target becomes more obvious as the relative hardness increases. By analyzing the two stages, the dynamic interaction and damage mechanism between the non-rigid projectile and the cement-based material target can be more deeply understood. This in-depth analysis provides a scientific basis for the design and material selection of the protective structure, which can promote the development of related engineering applications.

### Example 2

The values of the relative hardness of cement-based materials with unknown penetration resistance were obtained using the method described in Example 1. The relative hardness of each material obtained was substituted into the calculation formulas for the normalized penetration depth and the crater diameter in Example 1 to obtain the normalized penetration depth Y_{penetration depth} and the crater diameter Y_{crater diameter}. At the same time, the relative hardness was substituted into the calculation formulas for the projectile mass change, the projectile length change, and the projectile diameter change in Example 1 to obtain the normalized projectile mass change γₘ, the normalized projectile length change γₗ, and the normalized projectile diameter change γ_{d} obtained from the evaluation.

The penetration resistance of the sample in this example was actually measured according to the method in Example 1. The obtained penetration depth Y_{penetration depth}, crater diameter Y_{crater diameter}, normalized projectile mass change γₘ, normalized projectile length change γₗ, and normalized projectile diameter change γ_{d} were evaluated and found to be in good agreement with the actual measured data, indicating that the penetration resistance evaluation method obtained in this example can be applied to cement-based composite materials and penetrating projectiles with unknown properties.

### Example 3

The characteristic mechanism of projectile deformation is still unclear for the combination of the projectiles and the cement-based material target. For example, the scenario that an airplane collides with a concrete structure may be considered as an impact caused by a deformable projectile, and the actual problem requires a better understanding of the ability of concrete to resist the impact of the deformable projectile. The content in the present disclosure helps to fill the research gap in the impact of the deformable projectile on the cement-based material target, and helps to deeply understand the dynamic interaction and damage mechanism between the non-rigid projectile and the cement-based material target. This in-depth analysis provides a scientific basis for the design and material selection of the protective structure, which promotes the development of related engineering applications.

A car colliding with a flyover bridge pier is selected as an application case. When the car is equivalent to a homogeneous whole, it can be equivalent to a honeycomb aluminum material with pseudo Rockwell hardness of HRH 30-50. The predicted value of the Rockwell hardness is selected to be 50. According to the calculation method in Example 1, when REH is 1.2, the effective hardness of the material is 33.3. When the effective hardness of the flyover bridge pier material is higher than 33.3, the main form of impact damage is that the damage to the car is greater than the damage to the pier. When the effective hardness of the flyover bridge pier material is lower than 33.3, the main form of impact damage is that the damage to the car is less than the damage to the pier. Therefore, when the relative hardness is selected to be 1.2 during design, the two suffer less damage. When this calculation method is applied to penetration protection, the lower the relative hardness, the less damage to the protection project. When this calculation method is applied to striking a target, the higher the relative hardness, the better the impact effect on the target.

The foregoing descriptions are merely preferred embodiments of the present invention but are not intended to limit the present invention. A person skilled in art may make various alterations and variations to the present invention. Any modification, equivalent replacement, or improvement made within the spirit and principle of the present invention shall fall within the protection scope of the present invention.

1. A method for evaluating penetration resistance of a cement-based material against non-rigid projectiles, comprising:
S1: considering a cement-based material as a multiphase material, respectively measuring a Rockwell hardness of each phase, and calculating an equivalent length of each phase based on a volume ratio of each phase;
S2: determining an effective hardness of the cement-based material through weighted calculation based on the Rockwell hardness of each phase and the equivalent length of each phase;
S3: preparing a non-rigid projectile using a single-phase material as a parent material, and measuring a Rockwell hardness of the parent material as an effective hardness of the non-rigid projectile;
S4: calculating a ratio of the effective hardness of the non-rigid projectile to the effective hardness of the cement-based material to obtain relative hardness; and
S5: measuring penetration resistance and a projectile deformation degree for various cement-based material and non-rigid projectile combinations with different relative hardness, and respectively establishing association relationships between the penetration resistance and the relative hardness as well as between the projectile deformation degree and the relative hardness, wherein:
in S5, the penetration resistance comprises normalized penetration depth and crater diameter;
a relationship between the normalized penetration depth Y_{penetration depth} and the relative hardness REH is:
$${Y}_{crater diameter} = 74.56 - \frac{19893.505}{1 + {( \frac{REH}{0.120} )}^{0.120}} ;$$
a relationship between the crater diameter Y_{crater diameter} and the relative hardness REH is:
$${Y}_{crater diameter} = 74.56 - \frac{19893.505}{1 + {( \frac{REH}{0.120} )}^{0.120}} ;$$
in S5, the projectile deformation degree comprises normalized projectile length change, normalized projectile diameter change, and normalized projectile mass change;
a relationship between the normalized projectile mass change γₘ and the relative hardness REH is:
$${γ}_{m} = 1.229 + \frac{22859.691}{1 + {( \frac{REH}{0.361} )}^{9.307}} ;$$
a relationship between the normalized projectile length change γₗ and the relative hardness REH is:
$${γ}_{l} = 20.197 - \frac{217.6}{1 + {( \frac{REH}{0.947} )}^{8.544}} ;$$
a relationship between the normalized projectile diameter change γ_{d} and the relative hardness REH is:
$${γ}_{d} = 13.018 + \frac{6218.941}{1 + {( \frac{REH}{0.612} )}^{6.547}} .$$

2. The method for evaluating penetration resistance of a cement-based material against non-rigid projectiles according to claim 1, wherein in S1, considering the cement-based material as a three-phase material composed of a mortar matrix phase, a coarse aggregate phase and a fiber phase, obtaining a sample of the cement-based material, and measuring a Rockwell hardness of a surface of the sample as a Rockwell hardness of the mortar matrix phase; obtaining a coarse aggregate sample of the cement-based material, and measuring a Rockwell hardness of a surface of the coarse aggregate sample; and obtaining a fiber sample of the same material as fibers in the cement-based material, and measuring a Rockwell hardness of the fiber sample.

3. The method for evaluating penetration resistance of a cement-based material against non-rigid projectiles according to claim 2, wherein the mortar matrix phase comprises one or more of cement, silica fume, and fly ash; the coarse aggregate phase comprises 5-16 mm rock aggregate; and the fiber phase is one or more of straight copper-coated steel fibers, hooked-end copper-coated steel fibers, and organic fibers.

4. The method for evaluating penetration resistance of a cement-based material against non-rigid projectiles according to claim 1, wherein in S1, a volume ratio of the mortar matrix phase, the coarse aggregate phase and the fiber phase is calculated, and an equivalent length of the mortar matrix phase, an equivalent length of the coarse aggregate phase, and an equivalent length of the fiber phase are respectively obtained.

5. The method for evaluating penetration resistance of a cement-based material against non-rigid projectiles according to claim 4, wherein the equivalent length of each phase is obtained through a cube root of the volume ratio of each phase, and the equivalent length of the fiber phase is corrected according to the type of fibers.

6. The method for evaluating penetration resistance of a cement-based material against non-rigid projectiles according to claim 1, wherein in S2, in a weighted calculation method, a weight of an effective hardness of each phase is equal to a ratio of the equivalent length of the phase to a sum of the equivalent lengths of all phases.

7. The method for evaluating penetration resistance of a cement-based material against non-rigid projectiles according to claim 1, wherein in S3, a method for measuring the Rockwell hardness of the non-rigid projectile parent material sample is the same as a method for measuring the Rockwell hardness in S1.

8. The method for evaluating penetration resistance of a cement-based material against non-rigid projectiles according to claim 1, wherein in S5, an impact velocity is 390-430 m/s.
